(19) United States
(12) Patent Application Publication
(10) Pub. No.: US 2003/0073068 A1
(43) Pub. Date: Apr. 17, 2003

(54) Methods for application of tear-based biomarkers to ophthalmic product evaluation and clinical study management

(76) Inventor: Robert Edward Campbell, Chicago, IL (US)

Correspondence Address: Robert E. Campbell, 2456 N Geneva Terrace, Chicago, IL, 60614, US

(21) Appl. No.: 09/976,896

(22) Filed: Oct. 15, 2001

(51) Int. Cl. C12Q 1/00; G01N 33/53; G01N 33/537; G01N 33/543

(52) U.S. Cl. 435/4; 435/7.1

(57) ABSTRACT

Methods and test kits for characterizing and evaluating a mammal's ocular defenses and health are disclosed. More particularly, the invention concerns methods and test kits directed to measurements and subsequent comparative evaluations of tear constituents indicative of ocular defenses and health including antioxidants, antimicrobials, inflammatory mediators, and byproducts of cellular damage.

### CROSS-REFERENCE TO RELATED APPLICATIONS

Not Applicable

### STATEMENT REGARDING FEDERALLY SPONSORED RESEARCH OR DEVELOPMENT

No Federal funds were used in development of the present invention.

### REFERENCE TO A MICROFICHE APPENDIX

Not Applicable

### BACKGROUND OF THE INVENTION

1. Field of the Invention

The present invention relates to methods and test kits for assessing mammalian reactions to factors with ocular effect, including but not limited to ocular surgery, ophthalmic device use, and ophthalmic solutions and preparations. More particularly the present invention concerns methods and test kits directed to measurements and subsequent comparative evaluations of tear constituents indicative of ocular defenses or health for application in ophthalmic product evaluations and clinical study management.

2. Definitions

Unless defined otherwise, all technical and scientific terms and abbreviations used herein have the same meanings as is commonly understood by one skilled in the art to which this invention belongs. In order to provide a clear and consistent understanding of the specification and claims, including the scope to be given such terms, the following defined terms and definitions are provided.

| Term | Definition |
|---|---|
| “Additional | Characterization developed using Additional Data. |
| Characterization” | |
| “Additional Data” | OHV values and OHIs obtained from a mammal or group of mammals |
| | subsequent to Baseline Data. |
| “adverse event” | Any complication negatively impacting the health and function of the |
| | eye and related structures. |
| “Application” or | A specific application of a Product or Service, including but not limited |
| “Applications” | to, contact lens wearing modalities and/or periods, use of ocular |
| | surgical procedures for new classes of patients, use of ocular drugs |
| | for new or different indications. |
| “Baseline | Characterization developed using Baseline Data. |
| Characterization” | |
| “Baseline Data” | Initial measurement of the levels of OHVs, or measurement or |
| | evaluation of OHIs, present in a mammal or group of mammals taken |
| | prior to exposure to the subject of a Study. May include a series of |
| | measurements and/or evaluations. |
| “Benchmark | Refers to a Characterization that represents group or population |
| Characterization” | norms, for use as a comparative base for evaluation of Baseline |
| | Characterizations or Additional Characterizations. |
| “Characterization” | Refers to a mathematical, statistical, graphical, or other |
| | representation, translation, conversion, or modification of one or more |
| | mammal's OHV measurements, or OHI measurements or subjective |
| | evaluations. |
| “Ocular Health | An indicator of the health and function of the eye and related |
| Indicator” or “OHI” | structures, based on ophthalmic and/or systemic evaluations or |
| | measurements, including but not limited to measurement of |
| | intraocular pressure; evaluation of ocular anterior segment and |
| | adnexa, ocular media, ocular posterior segment; visual field |
| | screening; and/or results of systemic health screening and/or |
| | diagnostic tests for diseases and/or disorders that may have ocular |
| | manifestations. |
| “Ocular Health Vector” | Any tear constituent used or useful in the characterization or |
| or “OHV” | evaluation of a mammal's ocular defenses or health, including but not |
| | limited to antioxidants, antimicrobials, immune system mediators, |
| | inflammatory products or by-products, and byproducts of cellular |
| | damage or death. |
| “Participant” or | A mammal or group of mammals upon which ocular products are to |
| “Participants” | be or are being tested, or who act as controls, in a Study or Studies. |
| “Product” or “Products” | Any product with ocular application or effect, including but not limited |
| | to: contact lens materials; contact lens wetting, lubricating or |
| | disinfecting solution; ophthalmic solutions including antioxidant or |
| | decongestant drops; ophthalmic preparations, including steroid, anti- |
| | inflammatory or antimicrobial ointments. |
| “Service” or “Services” | Any service with ocular application or effect, including but not limited |
| | to surgical procedure methods equipment, and materials. |
| “Study” or “Studies” | Clinical, research, or other study or test, for regulatory approval or |
| | otherwise, of Products and/or Services and/or Applications. |
| “Subject” | One or more Products and/or Services under evaluation or test in a |
| | Study, i.e., the subject of a Study. |
| “US FDA” or “FDA” | United States Food and Drug Administration |
| “wearing modality” | Term referring to length of time a contact lens is worn. For example, |
| | daily wear refers to contact lens wear during waking hours; extended |
| | wear refers to 24-hour contact lens wear up to 7 days in total; |
| | continuous wear refers to 24-hour wear up to 30 days. |

3. Description of Prior Art

Current practice in ophthalmic clinical, research and other studies is to evaluate the impact of the subject of the Study (the “Subject”) on Participant ocular health through application of conventional screening and diagnostic methods and, if possible, Participant questionnaires. These methods also are employed in management of Participants and Study resources.

Potential Participants in a Study are initially screened for the presence or absence of an ocular or systemic disease or disorder, as dictated by sponsor goals and the Subject. Those that meet selection criteria are then monitored closely during the Study with strict adherence to Subject use/exposure criteria and periodic professional examinations. Typically, every effort is made to ensure that all Participants are followed through completion of the Study, or to a clinical outcome that indicates a threat to their vision or health. In the event Participants are human, periodic Participant evaluation of Subject performance is an important part of the Subject evaluation process and findings.

Conventional screening methods employed in such studies include Participant questionnaires, visual examination of the eyes and related structures, and measurement of various ocular and systemic parameters, including intraocular pressure, blood pressure, and blood glucose.

In the event an ocular or systemic disease or disorder is indicated from a Participant ophthalmic health screening during a Study, diagnostic testing may be pursued in an attempt to identify and treat the underlying condition. Such tests include evaluation of ocular surface condition via staining, measurement of tear flow rate, measurement of tear breakup time, and culturing samples for microbial identification.

Conventional screening and diagnostic methods are relatively complicated, time-consuming and expensive, as they each comprise a number of steps and require the judgment and opinion of a trained professional. In addition, since they require the presence of gross signs and symptoms for application, they require a relatively advanced state of disease or disorder.

While conventional methods of Subject evaluation and Study management generally are effective, reliance on gross outward signs and symptoms of ophthalmic distress and subjective Participant opinion on Subject performance can result in evaluations that, while demonstrating exceptional Subject performance, do not provide clear, objective data upon which to draw meaningful conclusions as to Subject performance in the Participant's general population, or how the Subject may compare to other competitive offerings in practice. In addition, while these studies can provide an adequate indication of important safety and efficacy issues in the event problems are severe or effect a large percent of the Participants, they also can miss issues which are less severe, impact a small percentage of the population, or present under less than perfect conditions. Finally, repeating the findings of a Study can be problematic, again due to the conventional reliance on subjective evaluations and personal opinion.

Participant perception of and response to discomfort or pain is an important factor in conventional Study methods, as it affects the screening and diagnostic processes. This can distort Study findings, as many patients will not complain of problems unless prompted, and then can vary widely in their description of symptoms and their severity. Unless the Participant develops outward signs or symptoms of an ophthalmic health problem, interpretation of Participant comment is difficult, resulting in potential Subject performance issues that are missed or lost in conflicting Participant descriptions.

Professional evaluations of Participant ophthalmic health are based on visual appraisal of the eye and related structures. Assessing gross outward signs and symptoms of ocular distress, including the presence and degree of redness, swelling, or discharge are key to the professional's determination of Participant ocular health. Such subjective assessments can and do vary from professional to professional, increasing variability in Study results and weakening application of Study results to a broader population.

An additional weakness of conventional ophthalmic screening and diagnostic techniques is that different ophthalmic health problems present with identical signs and symptoms. This can be an issue in Subject evaluation, as the underlying problem may be unrelated to the Subject, e.g., an allergic reaction to environmental factors may be attributed to the Subject.

Reliance on and subjective evaluation of gross signs and symptoms of ophthalmic distress can allow subclinical ophthalmic health issues to go unnoticed. A Participant with a transitory or chronic ophthalmic health issue may be considered to be in perfect ophthalmic health if the issue is at a subclinical level. Exposure to the Subject can exacerbate the health problem to a clinically detectable level, with the result that it is considered a Subject complication. Alternatively, the Subject can cause an ophthalmic health problem at the subclinical level that is missed in the evaluation process.

The lag between ocular insult and onset of observable signs and symptoms further complicates and obfuscates the Subject evaluation as currently conducted. This lag can be days, weeks or longer, depending on the type and degree of ophthalmic insult and individual Participant immune system reactivity. This can further distort Study findings, as at the time of pre-Study screening a prospective Participant may be developing an ophthalmic health problem that is not yet evident. When it does become evident, the problem will be erroneously attributed to the Subject. On the other hand, ophthalmic health issues triggered by the Subject may not manifest until after Study completion, and so be missed.

A further issue associated with current reliance on overt signs and symptoms of ophthalmic health problems is that they often appear with little or no prior indication. This results in less than optimal allocation of Study effort and resources, as all Participants must be examined periodically to detect their emergence. There simply is no way to differentiate between affected and unaffected Study Participants until adverse reactions become clinically apparent.

In addition, a Participant may encounter a “sudden onset” problem, e.g., a contact lens peripheral ulcer, which requires timely and appropriate action. Treating such emergency situations requires a higher level of Study personnel involvement and expense than if the underlying problem had been treated earlier, resulting in a relative waste of Study resources and unnecessary Participant discomfort. In addition, such complications can result in permanent Participant ocular damage.

In summary, current methods of evaluating the subject of an ophthalmic clinical, research or other study and Study management are limited by reliance on and interpretation of overt signs and symptoms of ocular distress, and subjective professional and Participant opinion on the effect or effectiveness of the subject of the Study.

### BRIEF SUMMARY OF THE INVENTION

The current invention employs measurement and comparison of the levels of tear-based biochemicals and other tear constituents (collectively, “Ocular Health Vectors” or “OHVs”) to characterize and evaluate the state of a mammal's ocular defenses and/or health for application in ophthalmic clinical, research, and other studies.

Specific tear constituents to be measured are chosen so that the state of a mammal's ocular defenses (antioxidants, antimicrobials, tear quality, etc.) and/or health (inflammatory mediators, byproducts of inflammation, indicators of cellular damage, etc.) can be characterized and evaluated according to the purposes of the Study.

Once OHVs of interest are identified, tear samples are taken from a mammal and tested to determine the level of each OHV. These measurements are then used to characterize the mammal's ocular defenses and/or health for comparison with other measurements from the same or different mammals. These comparisons provide the basis for evaluation of the state of, or change in state of, the mammal's ocular defenses and/or health.

Application of the present invention enables evaluation of the subclinical effects of Products or Services, both on a single mammal and on selected groups or populations of mammals. Such evaluations enable detailed, objective analyses and understandings of the ocular effect of Products or Services that are not possible using conventional methods.

For example, using the methods of the present invention the subclinical effect of a new extended wear contact lens material can be compared directly to previously FDA-approved extended wear materials to assess the relative impact of the two on the health and functioning of the eyes and related structures. Such a clinical study and associated analyses can demonstrate at what point in wear the new material is equivalent in ocular impact to the previously approved material. The findings of such a Study can justify FDA approval of contact lens wearing periods based on actual product performance, rather than arbitrary time periods, an outcome not possible using current ophthalmic Product evaluation methods.

As an additional example, the effect of two different ocular (or systemic) anti-inflammatory drugs on the ocular defenses and/or health of cataract patients can be compared either on an individual basis (e.g., sex and age matched Study Participants) or an overall population basis by applying results from a Product evaluation Study incorporating a statistically significant population of cataract patients. Current methods of Product and Service evaluation are limited in their ability to support such evaluations, as they are subject to bias from several sources and rely heavily on subjective measures of Product performance, making reliable comparisons and projections problematic.

The present invention overcomes limitations of current methods of Subject evaluation and Study management by eliminating reliance on subjective professional interpretation of overt signs and symptoms of ocular distress and Participant opinion on Subject performance. In addition, with its ability to detect subclinical ocular conditions, the present invention enables recognition of ocular issues that conventional methods miss.

Further benefits of the present invention include reduction or elimination of Study issues associated with both ambiguity of cause for overt signs and symptoms of ocular distress and subclinical ocular health issues. In addition, by enabling evaluation of the subclinical state of ocular health, or change in health, the present invention provides the ability to detect ocular health issues before they manifest, thereby reducing or eliminating issues associated with time lags between ocular insult and development of signs and symptoms and “sudden onset” events.

An advantage of the present invention is its flexibility in application to the needs and goals of diverse Studies through Study-specific OHV selection and tailoring of Product or Service evaluation methods and Study management techniques.

Use of measurable, objective, indicators of ocular defenses and health according to the methods of the present invention provide additional advantages to Product and Service evaluations. Such indicators enable detailed mathematical, statistical and graphical evaluations and representations of Subject performance. Performance can be evaluated on an absolute basis, i.e., based on changes in Participant ocular defenses and/or health, or on a comparative basis, i.e., compared directly to similar representations of other Products or Services to assess relative performance. In addition, use of measurable, objective factors also helps ensure that Study results can be reliably and accurately projected into larger populations. Current methods of Product and Service evaluation are limited in their ability to support such representations and projections due to their reliance on subjective evaluations and opinions.

Another advantage of the present invention is the ability to screen selectively and accurately prospective Participants according to the purpose of the Study, for example, to ensure all healthy Participants or, alternatively, Participants with a particular health issue. This application of the present invention helps ensure that issues unrelated to the purposes of the Study are avoided and that Study resources are more effectively employed.

Another advantage of the present invention is reduction of Study cost over Studies that rely upon application of conventional ophthalmic screening and diagnostic methods, in particular by utilizing lab technicians using rapid and inexpensive ELISA tests for OHV measurement versus time-consuming and expensive professional physical examinations.

A further advantage of the present invention is the ability to handle Participants differentially based on individual reaction to the Subject. To accomplish this, Participant subclinical reaction, positive or negative, to the Subject is evaluated using OHVs during the Study. The resulting detailed, objective record of subclinical changes in Participant ocular defenses or health enables the Study sponsor to allocate differentially Study effort and resources as the Study progresses according to individual Participant progression, rather than having to maintain the same level of Participant monitoring while waiting for development of overt signs and symptoms. This method of Study management as disclosed herein can result in additional focus on Participants who demonstrate reactions of interest, and reduction in monitoring of Participants with little or no reaction to the Subject, enabling more efficient and effective application of Study effort and utilization of Study resources.

These and other advantages and benefits will be apparent from the disclosures contained herein. Nowhere in the related art is there disclosed or suggested methods for evaluating the subject of an ophthalmic clinical, research or other study or managing such studies which anticipates the present invention.

### BRIEF DESCRIPTON OF THE SEVERAL VIEWS OF THE DRAWING

Not Applicable

### DETAILED DESCRIPTION OF THE INVENTION

The present invention employs measurement and comparison of the levels of tear-based biochemicals and other tear constituents (collectively, “Ocular Health Vectors” or “OHVs”) to characterize and evaluate the state of a mammal's ocular defenses and health for application in ophthalmic clinical, research, and other studies. The methods of the present invention enable, for the first time, the use of these subclinical indicators of the state of ocular defenses and health for the evaluation of Products and Services and the management of Studies.

Characterizations of the state of a mammal's ocular defenses or health as provided by the methods disclosed herein can be used to evaluate subclinical changes in a mammal's ocular defenses or health due to environmental factors, ocular surgery, ophthalmic medications, and other factors, e.g., to assess the ocular impact of contact lens wear or efficacy of ophthalmic preparations. Additionally, such characterizations may be compared to characterizations developed from other mammals for a variety of purposes, including screening a mammal for presence or absence of disease or disorder prior to inclusion in a Study, or evaluating a mammal's ocular defenses or health versus group or population norm.

Natural ocular defenses useful as OHVs include biochemicals important to tear film function, antioxidants, antimicrobials, and phagocytes. OHVs useful for characterizing and evaluating ocular health include inflammatory mediators and biochemicals associated with cellular injury or death.

Specific OHVs to be measured for characterization or evaluation of a mammal's ocular defenses or health are chosen to meet the needs and constraints of a particular Study. For example, a Study sponsor may wish to screen initially prospective Participants to focus on those with subclinical inflammation and “normal” (as determined by Study requirements) levels of natural ocular antimicrobials using two OHVs to keep costs low. Once the Study is underway, additional OHVs may be employed to provide a more detailed understanding of Participants' ocular defenses and health.

Table 1 below provides an overview of some of the more important tear constituents useful as OHVs, their role, and how their levels vary according to the health and functioning of the eye and related structures. Other or additional tear constituents may be employed in practice of the methods of the present invention, according both to the goals and purposes of the application to which the invention is directed, and the understandings of the presence and role of tear constituents at the time of practice. It is anticipated that the present invention will provide an efficient and effective means by which future understandings of the presence and role of tear constituents will be reduced to practical application.

TABLE 1
Example Selected Tear Constituents

| Tear | | |
|---|---|---|
| Constituent | Role | Indicates |
| Cystatin | Antimicrobial | Compromised ocular defenses if |
| (cysteine proteinase | | low. |
| inhibitor protein) | | |
| Fibronectin | Corneal wound healing; | Cellular damage/repair; |
| (glycoprotein) | Initially derived from plasma, | Increased vascular permeability |
| | then synthesized locally in the eye. | |
| GM-CSF | Inflammatory mediator | Allergic response, inflammation |
| (cytokine) | Prime PMN for enhanced chemotaxis, | |
| | superoxide generation and cytotoxic activity in | |
| | response to chemoattractants; | |
| | Enhances synthesis and release of DAF and | |
| | LTB₄ | |
| IgE | Inflammatory mediator | Elevated levels indicate |
| (glycoprotein) | | chronic/high inflammation due to |
| | | allergic reaction. |
| IgG | Inflammatory mediator | Elevated levels indicate |
| (glycoprotein) | | chronic/high inflammation |
| | | Sensitive to antigenic stimulation |
| Lactate | In combination with coenzyme NAD+ | Cellular damage. |
| dehydrogenase | catalyzes the interconversion of lactate and | Increased cell membrane |
| (enzyme) | pyruvate. | permeability due to hypoxia. |
| Lactoferrin | Antioxidant; | Compromised ocular defenses if |
| (iron binding protein) | Antimicrobial | low. |
| | Inflammatory mediator | If patient diagnosed with dry eye |
| | | then low level useful as |
| | | confirmatory test for aqueous |
| | | deficient dry eye |
| | | Lactoferrin is low in the following |
| | | conditions: |
| | | Aqueous dry eye; |
| | | Adenovirus conjunctivitis; |
| | | Herpes simplex; |
| | | Post-operative infection; |
| | | Protein malnutrition; |
| | | Trachoma |
| LTB₄ | Inflammatory mediator | Subclinical to clinical |
| (organic chemical) | Chemotactic for PMNs; | inflammation. |
| | Stimulates PMN degranulation, formation of | Originally from corneal epithelial |
| | ROS, arachidonic acid release, and | cells, stromal keratocytes and |
| | metabolism, | infiltrating PMNs; |
| LTC₄ | Inflammatory mediator | Subclinical to clinical inflammation |
| (organic chemical) | Increases vascular permeability, hyperemia, | associated with antigen response |
| | edema, mucous secretion | |
| Lysozyme | Antimicrobial | Compromised ocular defenses if |
| (bacteriolytic protein) | | low. |
| | | Generally varies with lactoferrin as |
| | | function of lacrimal gland function. |
| Polymorphonuclear | Antimicrobial | Increase with inflammation and |
| leukocytes | | associated chemotactic mediators; |
| RANTES | Inflammatory mediator—chemoattractant | Clinical or subclinical inflammation |
| (chemokine) | | Elevates in GPC |
| s-IgA | Antimicrobial | If low then patient at risk of |
| (glycoprotein) | Prevents bacterial binding to ocular surface; | infection |
| Tear lipocalin | Scavenges lipids in tear film, | Tear layer functionality; |
| (hydrophobic molecule | increases/maintains aqueous surface | Compromised ocular defenses if |
| binding proteins) | pressure; | low. |
| | Possible antimicrobial (cysteine proteinase | |
| | inhibitor) | |

Many scientific articles have appeared discussing the presence or absence of different tear-based biochemicals and other factors during the course of various ocular diseases and disorders, however, nowhere is there disclosed or suggested methods for evaluating the subject of a Study or managing a Study that anticipates the present invention.

Obtaining tear samples and measuring factors useful as OHVs is a straightforward process, well known to those skilled in the art. A number of companies provide equipment, supplies and technical support for measuring biochemical and other factors useful as OHVs, from complete test kits for a particular factor, to generalized test equipment, to components that may be used to create specialized or custom tests.

The following provides a brief overview of the OHV extraction and measurement process.

OHV measurements may be obtained from tear samples or extracted from materials in which they have been absorbed or to which they have become attached, e.g., contact lenses or absorbent paper.

Extraction of tear samples from the eye of a mammal for OHV measurements may be accomplished by a number of means, including micro capillary tube or use of absorbent material. Removing tear samples from absorbent material or other substrate also may be accomplished by a plurality of means, including centrifuge or via solvent.

Handling of such samples, e.g., additives for OHV stabilization and refrigeration, will depend upon the OHVs to be measured, available facilities, and form of measurement, among other factors. Suppliers of tests and equipment useful in measuring tear constituents have developed appropriate sample handling guidelines and solutions that they make available with their products.

Measurement of OHVs present in tear samples, raw or packaged in stabilizer, diluent or preservative, or as deposited on various substrates, or their extraction from a substrate and subsequent measurement may be accomplished by a plurality of means, including ELISA tests, HPLC, and dipsticks.

The selected OHV, resources available, level of accuracy required, equipment availability, and other application-specific factors will determine selection of an appropriate method of measuring an OHV level. Alternative methods of measurement are well known in the art, and are not essential to the present invention.

The invention relates to test kits providing means to practice the methods of the invention using materials and supplies directed to measurement of one or more OHVs of interest. The test kits and methods according to the present invention may contain all material necessary for tear sample acquisition and OHV measurement. Such test material may be specifically designed for measurement of one or more tear constituents.

Test kits may include materials for specific groups of OHVs, for example, antioxidants or inflammation-related OHVs, or combinations of OHVs that will enable full range of OHV measurements useful for characterizing and evaluating mammalian ocular defenses and health. Such test kits are contemplated to provide complete, convenient and relatively inexpensive means for OHV measurement.

Once the desired OHVs have been measured, they may be combined with other measures of ocular health, for example one or more characterizations, evaluations or findings from application of conventional ophthalmic screening or diagnostic methods (“Ocular Health Indicators”, or “OHIs”), to provide evaluations of ocular defenses or health according to needs and goals of the specific application of the methods of the invention.

To facilitate combined use of OHVs and OHIs, it is preferable that OHIs be expressed as values rather than in subjective terms. For example, bulbar hyperemia might be expressed on a scale of 1 to 4 rather than “none” or “severe”. Once this is accomplished, OHVs and OHIs may be mathematically adjusted, for example, weighted to reflect their relative importance for a particular application, or numerically combined to facilitate comparisons of overall ocular defenses and health.

The following discloses a general embodiment of the present invention, together with brief comments directing adaptation of the disclosed methods to the needs of a specific application of the present invention. The order of the steps of the disclosed method may be modified to facilitate application of the invention to the needs and constraints of different Studies, for example the steps of Paragraph 1 or Paragraph 2 may be reapplied after Baseline Data is obtained to refine the findings of the Study or adapt the Study to new or different constraints.

A general embodiment of the present invention comprises the following steps:

1. Determine which OHVs and OHIs are to be used, how they are to be obtained, and how they are to be measured.

This determination will be based on the purpose(s) of the application of the methods of the invention, available resources, ease of OHV measurement, and other factors specific to the particular application.

For example, comparison of the ocular impact of two different contact lens materials likely would include both antimicrobial and inflammatory mediator OHVs. On the other hand, evaluation of an ophthalmic solution for relief of dry likely would employ antioxidant and inflammatory mediator OHVs.

If choice of OHV is constrained by available resources, a minimal number of OHVs may be employed, and then only those for which commercially available ELISA tests are available, for example lactoferrin (Touch Scientific, Raleigh, N.C.) to evaluate antimicrobial defenses, and IL-8 (R&D Systems, Minneapolis, Minn.) and LTB₄ (Perspective Diagnostics, Cambridge, Mass.) to evaluate inflammatory state.

Alternatively, if particular OHV is required, for example the presence of s-IGA antibodies against Pseudomonas aeruginosa, development of a specialized test may necessary, for example an ELISA.

2. Determine how the OHVs and OHIs to be obtained are to be characterized.

Characterization comprises the process of mathematically, statistically, graphically, or otherwise representing, translating, converting or modifying OHV or OHI measurements or evaluations. This process does not provide any understanding of what the measured levels mean, it merely puts them in a form in which they can be compared to other measured levels as part of the evaluation process.

For the purposes of the disclosure of the present invention made herein, particularly to provide for simplification of such disclosure, all OHV and OHI measurements and evaluations are considered characterized prior to any evaluation of ocular defenses or health, even if such characterization step is, for example, multiplication of a measured value by one (1).

The particular mathematical adjustment made to any or all of the OHVs and OHIs in practice of the present invention will depend upon, among other things, which ones are employed, their relative importance to the application, and how the measurements are to be used. Accordingly, the present invention is not limited to the use of unadjusted OHVs and OHIs, and is intended to encompass all manipulations of OHV and OHI values, however obtained.

For example, the logarithm of inflammatory mediators may be used, antioxidants may be represented by their absolute values multiplied by a factor to give them equal weight with inflammatory mediators, and epithelial edema may be represented on a scale of 0 to 4 to reflect severity, then multiplied by a scale factor.

In addition to mathematical manipulation of OHVs and OHIs, statistical and graphical representations may be developed for comparative purposes. For example, the mean and standard deviation of the levels of selected antioxidant and inflammatory mediators for hydrogel contact lens patients with 6 months or more of wearing time may be used to evaluate a mammal's response to contact lens wear.

If the intent is to obtain indications of ocular defenses or health at one or more points in time for comparative purposes, individual values of OHVs and OHIs may be summed to arrive at single representative measures of ocular defenses or health for each point.

Alternatively, if changes in OHVs and OHIs are to be determined over a period of time or ocular exposure to stress, stimuli, medication, or other factors, changes in individual tear constituents over appropriate periods may first be calculated, and then summed to arrive a single representative measure. Note that these changes may be used directly, e.g., to provide an indication of the rate of change in ocular defenses or health, or they may be compared to changes experienced by other individuals, e.g., to predict an outcome.

For example, using group mean and standard deviation may facilitate identification of an individual to a group with known characteristics. In this case, a composite measure within, e.g., 1 standard deviation of the group mean would indicate equivalence with the group. An example of this would be selection of Participants for a clinical study where all Participants need to have an average level of ocular defenses or health. Comparing Participants to a group determined to be “average” after extensive testing would accelerate screening and lower costs, as it would not be necessary to conduct a rigorous ocular examination on all Participants.

Alternatively, graphical characterizations may be used to accomplish similar goals.

3. Obtain Baseline Data. These measurements or evaluations will be obtained prior to exposure to a subject of the Study.

(a) Take one or more samples of tear fluid from one or both eyes of the mammal; and

(b) Measure the levels of selected OHV(s) in said tear fluid sample(s); and

(c) Obtain measurements or subjective evaluations of OHIs.

4. Develop Baseline Characterizations.

These characterizations will be performed according to the decisions made in Paragraph 2, above, as modified based on study findings or other factors that may have arisen since initial decisions were made.

5. Evaluate the mammal's ocular defenses or health using Baseline Characterizations, as appropriate to the purposes of the evaluation.

The process of evaluating a mammal's ocular defenses and/or health on the basis of Baseline Characterizations requires comparison of a Characterization to identical or similar Characterization(s) that represent a known state(s) of ocular defenses and/or health.

For example, comparing a mammal's Characterization with the average of Characterizations obtained from normal, healthy mammal's provides an indication of the mammal's ocular defenses and/or health relative to its peers. This evaluation is useful, for example, in screening mammals for inclusion in a Study.

6. Obtain Additional Data at appropriate intervals, for example, after the lapse of time, or after period of wear or use of an ocular Product.

(a) Take one or more samples of tear fluid from one or both eyes of the mammal; and

(b) Measure the levels of selected OHV(s) in said tear fluid sample(s); and

(c) Obtain measurements or subjective evaluations of OHIs.

7. Develop Additional Characterizations.

These characterizations also will be performed according to Paragraph 2, above, as modified based on study findings or other factors that may have arisen since initial decisions were made.

8. Evaluate the mammal's ocular defenses or health using Baseline Characterizations and/or Additional Characterizations, as appropriate to the purposes of the evaluation.

Comparing an Additional Characterization of a mammal's ocular defenses or health to a Base Characterization or other Additional Characterizations from the same mammal enables an evaluation of changes in the mammal's ocular defenses and/or health. For example, a mammal may have experienced a decrease in ocular antimicrobials after exposure to an ophthalmic steroid, indicating a decrease in natural ocular defenses.

Comparison of an Additional Characterization to an Additional Characterization(s) from other mammals provides an indication of relative ocular defenses or health, e.g., to determine which Participants in a study have the strongest reaction to the subject of a clinical study, or how such mammals compare to selected groups or populations of mammals.

Those skilled in the art will realize that the disclosed general method of the present invention, and variations thereof, have a great number of applications. Based on the disclosure herein, those skilled in the art will be able to make necessary adjustments to the general method of the invention to suit other applications.

The following embodiments of the invention build upon the disclosed general method, and are intended only to provide examples of application of the invention to several important needs and are not intended to limit the scope of the present invention in any way.

Product Evaluations and Comparisons

Alternative embodiments of the present invention provide methods to evaluate or compare the ocular effect of ocular surgical procedure methods, equipment, or materials; wear or use of different ocular devices or products; exposure to different applications of ocular devices or products, or products with ocular effect; and other Products or Services.

Applications for these methods include, but are not limited to, evaluation of new contact lens material performance, or comparisons of new contact lens materials to previously US FDA-approved and accepted materials, for example, as a means to justify US Food and Drug Administration approval of a wearing periods for a new contact lens material, and of the effectiveness, or relative effectiveness, of ophthalmic drugs and preparations for use by patients with ocular surface disease.

One embodiment of the invention directed to such evaluations and comparisons uses individual changes in ocular defenses or health as the basis for Product or Service performance. This embodiment comprises the steps of:

1. Determine which OHVs and OHIs are to be used, how they are to be obtained, and how they are to be measured.

2. Determine how the OHVs and OHIs to be obtained are to be characterized.

3. In the event Participants were not screened using the methods of the present invention, optionally obtain Baseline Data:

(a) Take one or more samples of tear fluid from one or both eyes of the mammal; and

(b) Measure the levels of selected OHV(s) in said tear fluid sample(s); and

(c) Obtain measurements or subjective evaluations of selected OHIs.

4. Develop Baseline Characterizations.

5. Evaluate the mammal's ocular defenses or health using Baseline Characterizations, as appropriate to the purposes of the evaluation.

For Participants who were not screened prior to enrollment using the methods of the present invention, it may be desirable to evaluate their ocular defenses or health prior to exposure to a subject of the Study, for example by comparing Baseline Characterizations to a Characterization developed from a group of mammals considered to have normal levels of ocular defenses and health. This enables comparisons with subsequent Characterizations and can provide additional insights into the ocular impact of a subject of the Study.

6. Obtain Additional Data at appropriate intervals, for example, after the lapse of time, or after periods of exposure to a subject of the Study. If Baseline Data was not obtained, the first of these Additional Data will provide a similar function.

(a) Take one or more samples of tear fluid from one or both eyes of the mammal; and

(b) Measure the levels of selected OHV(s) in said tear fluid sample(s); and

(c) Obtain measurements or subjective evaluations of selected OHIs.

7. Develop Additional Characterizations.

8. Compare Baseline and Additional Characterizations to each other to evaluate the ocular effect of a subject of the Study on the Participant. In the event the relative effect of Products or Services is desired, compare Baseline and Additional Characterizations across such Products or Services.

In this case, changes in a Participant's OHVs, as indicated by Additional Characterizations, provide a measure of the ocular effect of a subject of the Study. Significant changes may indicate an increase in risk of adverse events or complications from exposure to a subject of the Study, or, conversely, an improvement in ocular defenses or health.

Combining Characterizations from some or all the Participants in the Study in accordance with statistical precepts and methods may provide an indication of the ocular effect of a subject of the Study on the overall group or population of such mammals.

Comparing Characterizations obtained from mammals exposed to different Products or Services enables objective and repeatable evaluations of the relative effect of different Products or Services.

An alternative method of Product or Service evaluation or comparison contrasts individual Characterizations against Characterizations obtained from a group, for example, to assess the effectiveness of a Product on a particular group of mammals. An example of this is the evaluation of the effectiveness of an antioxidant eye drop on dry eye patients. In this case, Additional Characterizations obtained from a mammal initially screened for the presence of dry eye may be compared to Characterizations obtained from dry eye patients.

A further example of this type of Product or Service evaluation or comparison is identification of individuals or groups with different reactions to a Product or Service. In this case, OHVs and OHIs may be obtained only after exposure to the subject of the Study, not necessarily both, as the Study may only be interested in final levels rather than changes in levels. Evaluating Product performance, or relative performance, on different demographic groups may then be accomplished using this embodiment of the present invention.

This method provides a means for evaluating the effect of a Product or Service on individual mammals or groups, as well as a means of comparing the ocular effects of Products or Services

One embodiment of this method of the invention comprises the steps of:

1. Determine which OHVs and OHIs are to be used, how they are to be obtained, and how they are to be measured.

2. Determine how the OHVs and OHIs to be obtained are to be characterized.

3. In the event Participants were not screened using the methods of the present invention, optionally obtain Baseline Data:

(a) Take one or more samples of tear fluid from one or both eyes of the mammal; and

(b) Measure the levels of selected OHV(s) in said tear fluid sample(s); and

(c) Obtain measurements or subjective evaluations of selected OH Is.

4. Develop Baseline Characterizations.

5. Evaluate the mammal's ocular defenses or health using Baseline Characterizations, as appropriate to the purposes of the evaluation.

For Participants who were not screened prior to enrollment using the methods of the present invention, it may be desirable to evaluate their ocular defenses or health prior to exposure to a subject of the Study, for example by comparing Baseline Characterizations to a Characterization developed from a group of mammals considered to have normal levels of ocular defenses and health. This enables comparisons with subsequent Characterizations and can provide additional insights into the ocular impact of a subject of the Study.

6. Obtain Additional Data at appropriate intervals, for example, after the lapse of time, or after periods of exposure to a subject of the Study. If Baseline Data was not obtained, the first of these Additional Data will provide a similar function.

(a) Take one or more samples of tear fluid from one or both eyes of the mammal; and

(b) Measure the levels of selected OHV(s) in said tear fluid sample(s); and

(c) Obtain measurements or subjective evaluations of selected OHIs.

7. Develop Additional Characterizations.

8. Compare Baseline and Additional Characterizations to each other and to Characterizations obtained from other mammals to evaluate the effect, or relative effect, of the Product or Products on the mammal's ocular defenses or health.

Clinical Study Management

The present invention may be applied both in initial screening and subsequent monitoring of Study Participants.

When applied to initial screening of prospective Participants, the present invention ensures selection of mammals with a particular desired state of ocular defenses or health. While “free from ocular disease” often is specified, some Studies require specific problems, e.g., a Study of a new ophthalmic preparation may require dry eye patients, or wish to include individuals with “normal” (i.e., not necessarily free from ocular disease), ocular defenses or health.

This application of the present invention enables more objective means of Participant screening, using OHVs, than otherwise possible, while also adding additional definition and refinement to the screening process. In the case of a dry eye patient requirement, for example, the Study sponsor may screen for dry eye patients with low antioxidant and high inflammatory tear constituents, indicating a relatively severe stage of this ocular surface disease.

One embodiment of the present invention, as disclosed below, compares Characterization(s) of prospective Participant's ocular defenses and/or health against those of a specific group or population. This application of the present invention enables Participant selection using criteria based on specific group characteristics such as disease state, sex, and age.

An embodiment of the invention directed to this application comprises the following steps:

1. Determine which OHVs and OHIs are to be used, how they are to be obtained, and how they are to be measured.

2. Determine how the OHVs and OHIs to be obtained are to be characterized.

3. Obtain initial pre-Study measurements of OHVs and measurements or subjective evaluations of OHIs, if any. For those prospective Participants who are selected for enrollment in the Study, these measurements and/or evaluations will comprise Baseline Data.

(a) Take one or more samples of tear fluid from one or both eyes of the mammal; and

(b) Measure the levels of selected OHV(s) in said tear fluid sample(s); and

(c) Obtain measurements or subjective evaluations of selected OHIs.

4. Characterize selected pre-Study OHV or OHI data.

5. Evaluate the mammal's ocular defenses or health using Characterizations derived from pre-Study OHVs or OHIs to determine the mammal's inclusion in the Study.

Compare Characterizations derived from pre-Study OHVs or OHIs to Characterizations of identical or similar OHVs or OHI data taken from mammals representative of the desired state of ocular defenses or health to determine whether the individual fits the chosen ocular criteria and should be enrolled in the Study.

Once Participants have been selected, the present invention enables screening of Participants during the course of the Study for differential handling. Such screening may compare Participant pre-Study (Baseline Characterizations) and interim Characterizations (Additional Characterizations), to evaluate Participant reaction to the subject of the Study.

With the understandings gained, Participants may be handled differentially, enabling more effective use of Study resources than possible using conventional methods of Study management, for example, Participants with little or no reaction to the subject of the Study may be monitored less frequently than those with strong reactions.

An embodiment of the invention directed to this application comprises the following steps:

1. Determine which OHVs and OHIs are to be used, how they are to be obtained, and how they are to be measured.

2. Determine how the OHVs and OHIs to be obtained are to be characterized.

3. In the event Participants were not screened using the methods of the present invention, optionally obtain Baseline Data:

(a) Take one or more samples of tear fluid from one or both eyes of the mammal; and

(b) Measure the levels of selected OHV(s) in said tear fluid sample(s); and

(c) Obtain measurements or subjective evaluations of selected OH Is.

4. Develop Baseline Characterizations.

5. Evaluate the mammal's ocular defenses or health using Baseline Characterizations, as appropriate to the purposes of the evaluation.

For Participants who were not screened prior to enrollment using the methods of the present invention, it may be desirable to evaluate their ocular defenses or health prior to exposure to a subject of the Study. This enables comparisons with subsequent Characterizations and can provide additional insights into the ocular impact of a subject of the Study.

6. Obtain Additional Data at appropriate intervals, for example, after the lapse of time, or after periods of exposure to a subject of the study. If Baseline Data was not obtained, the first of these Additional Data will provide a similar function.

(a) Take one or more samples of tear fluid from one or both eyes of the mammal; and

(b) Measure the levels of selected OHV(s) in said tear fluid sample(s); and

(c) Obtain measurements or subjective evaluations of selected OHIs.

7. Develop Additional Characterizations.

8. Compare Baseline and Additional Characterizations to each other to evaluate the reaction of the Participant to a subject of the Study and to determine whether to continue the Participant in the Study and, if so, on what basis.

For example, a Participant may have a severe subclinical reaction to a Product without clinical signs or symptoms being evident. In this case, the Participant may either be followed more closely, to avoid any serious complication, or removed from the Study.

Alternatively, a Participant may display no subclinical reaction to a Product after a period of exposure, while others in the Study have fully developed reactions. In this event, the Participant may be considered unaffected by the Product and monitored less frequently than others or dropped from the study.

An alternative embodiment of the invention useful for screening Participants for differential handing in a Study evaluates a mammal's Additional Characterizations to Characterizations from other mammals. Comparative individuals, or groups, may be Participants in the Study themselves, for example, if identification of those only with the most extreme reactions is desired, or from outside the Study, for example, in the event variance from a defined group is desired.

This embodiment of the invention comprises the following steps:

1. Determine which OHVs and OHIs are to be used, how they are to be obtained, and how they are to be measured.

2. Determine how the OHVs and OHIs to be obtained are to be characterized.

3. In the event Participants were not screened using the methods of the present invention, optionally obtain Baseline Data:

(a) Take one or more samples of tear fluid from one or both eyes of the mammal; and

(b) Measure the levels of selected OHV(s) in said tear fluid sample(s); and

(c) Obtain measurements or subjective evaluations of selected OH Is.

4. Develop Baseline Characterizations.

5. Evaluate the mammal's ocular defenses or health using Baseline Characterizations, as appropriate to the purposes of the evaluation.

For Participants who were not screened prior to enrollment using the methods of the present invention, it may be desirable to evaluate their ocular defenses or health prior to exposure to a subject of the Study. This enables comparisons with subsequent Characterizations and can provide additional insights into the ocular impact of a subject of the Study.

6. Obtain Additional Data at appropriate intervals, for example, after the lapse of time, or after periods of exposure to a subject of the Study. If Baseline Data was not obtained, the first of these Additional Data will provide a similar function.

(a) Take one or more samples of tear fluid from one or both eyes of the mammal; and

(b) Measure the levels of selected OHV(s) in said tear fluid sample(s); and

(c) Obtain measurements or subjective evaluations of selected OH Is.

7. Develop Additional Characterizations.

8. Compare Baseline and Additional Characterizations to each other and to Characterizations obtained from other mammals to evaluate the relative reaction of the Participant to a subject of the Study and to determine whether to continue the Participant in the Study and, if so, on what basis.

Once Participants have been identified according to their reactions to the subjects of the Study they may be handled differentially according to study purposes and goals, as discussed above. For example, in evaluating a new wearing period for a particular contact lens material, those with little or no changes in antioxidants, inflammatory mediators, or indicators of cellular damage after 3 to 4 months of wear may be discontinued from the study, while those with decreases in antioxidants, increases in inflammatory mediators and indicators of cellular damage may remain in the study so that the study sponsor may assess the incidence of adverse events associated with the prospective wearing period.

An important aspect of the present invention is its flexibility in application to Study management.

For example, the present invention may be configured to meet the needs of a Study to evaluate the effect of a medication on cataract patients, and so may be confined to post-surgery OHV measurements and associated Additional Characterizations that provide indications of improving ocular defenses or health, e.g., increasing antioxidant levels, decreasing inflammatory byproducts and decreasing indicators of cellular damage.

At the same time, the present invention also is applicable to Studies of contact lens wearing periods, where deterioration in ocular defenses or health is important. In this case, both Baseline and Additional Characterizations are important to detect, for example, decreases in antimicrobial levels, increases in inflammatory mediators and indicators of cellular damage that take place.

Those skilled in the art will realize that modifications to the methods disclosed for application of the present invention to the area of Study management, as well as other and further embodiments can be made to suit individual Study requirements without departing from the spirit of the invention, and it is intended to include all such modifications and other and further embodiments as come within the true scope of the invention disclosed herein.

Group/Population Characterizations

An additional embodiment of the present invention provides a method to Characterize ocular defenses or health for groups or populations of mammals. Such Characterizations may be used as benchmarks against which to compare individuals or groups of individuals for evaluation of their ocular defenses or health. These Benchmark Characterizations may be developed before or after mammalian exposure to Products or Services.

Examples of groups for which such characterizations would be useful include:

1. Mammals known to have a common response to one or more factors with ocular effect. For example, the effectiveness of an ophthalmic preparation for the relief of ocular allergic disease.

2. Normal individuals in a population, where the group was selected at random to represent accurately the distribution of selected OHVs in such population. In this case, all mammals in the benchmark Characterization are not necessarily healthy, and so the Characterization reflects average levels of ocular defenses and health. This benchmark Characterization would have application in Studies of, e.g., the impact of new contact lens materials on the general population. Given the presence of pre-existing conditions, this benchmark requires a relatively large number of Participants to reduce possible bias due to any one member.

3. Individuals who are known to be free of ocular or systemic disease or disorder, to represent an ideal level of selected OHVs. This benchmark would be useful in Studies with small numbers of Participants, where the potential bias in Study findings introduced by a single Participant with a preexisting subclinical condition is high.

4. Individuals who are known to have a particular disease or disorder. This benchmark is useful, for example, where the subject of the Study is targeted to specific groups, e.g., cataract patients.

An embodiment of the present invention directed to development of such Benchmark Characterizations comprises the steps of:

1. Determine which OHVs and OHIs are to be used, how they are to be obtained, and how they are to be measured.

2. Determine how the OHVs and OHIs to be obtained are to be characterized.

3. In the event Characterization of the group or population is desired to reflect normal conditions, obtain Baseline Data prior to exposure to any factor with ocular effect:

(a) Take one or more samples of tear fluid from one or both eyes of the mammals to be included in the groups or population; and

(b) Measure the levels of selected OHV(s) in said tear fluid sample(s); and

(c) Obtain measurements or subjective evaluations of selected OHIs.

4. Develop Baseline Benchmark Characterizations for the mammals in the group or population, with the final form being one or more composite Baseline Benchmark Characterizations representative of the group or population.

These Baseline Benchmark Characterizations should be developed using statistical precepts and methods well known to those skilled in such art so that they accurately and reliably represent the group or population of interest.

5. In the event Characterization of the group or population is desired to reflect ocular defenses or health after exposure to a factor with ocular effect, obtain Additional Data after such exposure at appropriate intervals, for example, after the lapse of time, or after periods of exposure to the factor with ocular effect.

(a) Take one or more samples of tear fluid from one or both eyes of the mammal; and

(b) Measure the levels of selected OHV(s) in said tear fluid sample(s); and

(c) Obtain measurements or subjective evaluations of selected OHIs.

6. Develop Additional Benchmark Characterizations for the mammals in the group or population, with the final form being one or more composite Additional Benchmark Characterizations representative of the group or population.

Additional Benchmark Characterizations should be developed using statistical precepts and methods well known to those skilled in such art so that they accurately and reliably represent the group or population of interest.

### EXAMPLE

Multiple examples of application of the present invention to the areas of Product and Service evaluation and comparison, and Study management have been provided in conjunction with the disclosure herein of various embodiments of the invention.

The following provides an example of application of the present invention to a clinical study for FDA submission comparing the ocular impact of 7-day extended wear hydrogel contact lens wear (“EW”) to 30-day continuous wear silicon-hydrogel wear (“CW”).

Study protocol dictates a crossover analysis whereby Participants use EW lenses for a period of 2 months, then CW lenses for 6 months.

Screening prospective Participants for study enrollment is accomplished using data collected at the initial interview. These data then comprise the Baseline Data and associated Baseline Characterization for enrolled Participants.

Participant data is obtained at the end of each wearing period (regardless of length, with Participant cooperation) for the EW lenses until day 60. At day 60 final EW Additional Data and Additional Characterizations are obtained and Participants switched to CW lenses.

Once in CW lenses Participants are tested weekly for the first month (month 3 of the study), then at the end of each wear period (regardless of length, with Participant cooperation) for 5 months (through month 8 of the study).

In excess of five hundred Participants are selected from the population of healthy EW patients who have worn their lenses between 6 and 9 months. The clinical study is conducted at 14 investigational sites in eight geographically diverse metropolitan areas.

The following highlights application of the present invention to various aspects of the study:

It is determined prior to start of the study (as part of Study Protocol development) that Participants will be screened for antioxidant, antimicrobial, inflammatory and allergic OHVs. In addition, professional evaluations of Participant edema, corneal neovascularization, corneal staining, bulbar hyperemia, conjunctival response, and evidence of complications are to be graded from 0 to 4 for use as Other Health Indicators.

Characterization of OHVs and OHIs are to be performed using mathematical and statistical methods. Study findings include OHVs and OHIs both separately and in combination. Graphical representations of OHVs and OHIs are completed for comparative and evaluative purposes.

Initial Screening

Initial interviews of prospective Participants are conducted at each of the investigational sites. A written questionnaire and Consent Form are completed by each prospective Participant to document relevant issues, including time in EW lens, and satisfy informed consent requirements.

Tear samples are taken at the initial visit after completion of documentation. Sample extraction is via micropipette. Total sample size is approximately four microliters from each eye. Tear samples are stabilized and diluted according to OHV test requirements.

Tear samples are tested at each investigational site by trained technicians. Both commercially available and custom ELISA tests are employed in sample testing. Such tests are packaged in kit form, with a single kit containing sufficient materials and supplies to test a single individual. Professional slit lamp examination is completed and results documented.

Tear sample and quantified (scale of 0 to 4) professional evaluations are sent daily from each investigational to a central location for Characterization and evaluation.

In order to screen prospective Participants for enrollment in the study, normal levels of OHVs must be defined against which they may be screened. Contact lens wear provokes an ocular reaction that affects the levels of natural antioxidant, antimicrobial, inflammatory, and allergic tear constituents. Selecting Participants from the population of “healthy” EW patients, therefore, means that selection criteria must reflect OHV levels normal for successful EW contact lens patients.

Given the size and geographic distribution of the subject study, development of appropriate screening criteria, in the form of a Benchmark Characterization, is possible using data obtained at the screening interviews. In the case of a smaller study, a Benchmark Characterization may have to be developed from mammals outside the study.

Accumulation and statistical analysis of prospective Participant Baseline (screening) Data is accomplished as it arrives at the central location. Combining such Data, then statistically analyzing it to identify norms and ranges for the Participant population develop the Benchmark Characterization.

In the case of the subject study, only OHV Characterizations are employed for screening, as OHI data and associated Characterizations do not provide useful additional information. With the development of the Benchmark Characterization, criteria for study enrollment are set and Participants screened and enrolled.

Differential Handling

Participants return regularly throughout the study to their investigational sites for testing. Length of time of lens wear and subjective comments are documented, and then tear samples obtained and tested, and slit lamp examination performed. Results are sent to the central location for Characterization and evaluation.

Participant OHV Additional Data and associated OHV Additional Characterizations obtained through the course of the study are evaluated to adjust Participant monitoring, i.e., Participants who display deterioration in their ocular defenses or health, as evidenced by a decrease in antioxidant or antimicrobial factors, or an increase in inflammatory or allergic factors, are followed more closely than others. This proactive Participant management reduces the risk of adverse events and associated Participant discomfort and use of study resources.

In a different or longer Study, Participant evaluations can be used to reduce or eliminate Participant monitoring for those who display little or no adverse reaction to the subject of the Study.

Comparative Product Evaluation

Upon study completion, OHV Baseline and OHV Additional Characterizations for EW use are compared to those for CW (the final EW OHV Additional Characterization being the CW OHV Baseline Characterization), both on a per-Participant and group basis. Additional comparisons include OHV Characterizations obtained at the end of the EW period to those obtained at the end of the CW period, and the Benchmark OHV Characterization versus a comparable treatment of CW OHV Additional Data obtained at the end of the CW period, to evaluate how Participant and group norms and ranges have shifted.

The different ocular effects of the two lens materials and wearing modalities are evaluated on the basis of impact on ocular defenses and health, where relatively higher levels of ocular antioxidants or antimicrobials or lower levels of ocular inflammatory or allergic factors indicate a material and wearing modality with relative health benefits.

As can be seen from the disclosures contained herein, an important strength of the present invention is that it may easily be adapted to a wide range of Studies. Decisions pertaining to specific application of the present invention, including determination of which OHVs to employ, whether and which OHIs to include, how to best characterize OHVs or OHIs for evaluative purposes, and the conclusions to draw from specific comparisons, are determined by the specific application of the present invention, Study resources and constraints, and individual judgment.

Criteria used to determine the application and meanings of OHVs depend on the purposes and goals of use of the methods of the present invention. The present invention contemplates the use of sound professional judgment in its application to a particular use, and it is intended to include all such criteria as they may be derived from practice of the present invention.

The invention has been described in detail, with reference to certain embodiments, in order to enable the reader to practice the invention without undue experimentation. Theories of application and use of the present invention have been offered to better enable the reader to understand the invention, but such theories do not limit the scope of the invention.

A person having ordinary skill in the art will readily recognize that many of the previous components, compositions, and parameters may be varied or modified to a reasonable extent and that modifications and other and further embodiments can be made without departing from the scope and spirit of the invention and it is intended to include all such further modifications and other and further embodiments as come within the true scope of the claims set forth herein.

Furthermore, titles, headings, example materials or the like are provided to enhance the reader's comprehension of this document, and should not be read as limiting the scope of the present invention. Accordingly, the intellectual property rights to the invention are defined by the following claims, reasonable extensions and equivalents thereof, and interpreted in view of the disclosure herein.

I claim:

1. A method for characterizing and evaluating the effect of one or more products or services on the ocular defenses or health of one or more mammals in a clinical, research or other study comprising:
(a) optionally taking one or more samples of tear fluid from one or both eyes of a mammal before exposure to a product or service under study; and
(b) taking one or more samples of tear fluid from one or both eyes of a mammal after exposure to a product or service under study; and
(c) measuring the levels of one or more tear constituents in said tear fluid sample(s); and
(d) using the measured levels of tear constituent(s) to characterize and evaluate the effect of a product or service under study on the ocular defenses or health of one or more mammals in the study.

2. A method for screening mammals for inclusion in a clinical, research or other study comprising:
(a) taking one or more samples of tear fluid from one or both eyes of a mammal prior to inclusion in the study; and
(b) measuring the levels of one or more tear constituents in said tear fluid sample(s); and
(c) using the measured levels of tear constituent(s) to characterize and evaluate the mammal's ocular defenses or health and to aid in determining the mammal's inclusion in the study.

3. A method for screening mammals for differential handling in a clinical, research or other study comprising:
(a) optionally taking one or more samples of tear fluid from one or both eyes of a mammal before exposure to a product or service under study; and
(b) taking one or more samples of tear fluid from one or both eyes a mammal after exposure to a product or service under study; and
(c) measuring the levels of one or more tear constituents in said tear fluid sample(s); and
(d) using the measured levels of tear constituent(s) to characterize and evaluate the mammal's ocular defenses or health and to aid in determination of whether to continue the mammal in the study and, if so, on what basis.

4. A method for characterizing the ocular defenses or health of one or more mammals for use as a comparative base, comprising:
(a) optionally taking one or more samples of tear fluid from one or both eyes of a mammal before exposure to one or more selected products or services; and
(b) optionally taking one or more samples of tear fluid from one or both eyes of a mammal after exposure to one or more selected products or services; and
(c) measuring the levels of one or more tear constituents in said tear fluid sample(s); and
(d) using the measured levels of tear constituent(s) to characterize the ocular defenses or health of one or more mammals.

5. The method of claim 4 wherein one or more mammals are selected to be representative of mammals: generally in a population; in good health in a specific population; from specific demographic groups; suffering from specific diseases or disorders; or otherwise, according to the purposes of comparison.

6. The method of claim 1, 2, 3, or 4 wherein a product or service to be studied or under study has an effect on the eyes or related structures.

7. The method of claim 1, 2, 3, or 4 wherein a product to be studied or under study is contact lenses.

8. The method of claim 1, 2, 3, or 4 wherein a service to be studied or under study is a surgical vision correction procedure.

9. The method of claim 1, 2, 3, or 4 wherein a product to be studied or under study is a contact lens wetting, lubricating, disinfecting or storing solution.

10. The method of claim 1, 2, 3, or 4 wherein a product to be studied or under study is an ophthalmic solution or preparation.

11. The method of claim 1, 2, 3, or 4 wherein the purpose of the study is to compare the effects, or relative effects, of two or more products or services.

12. The method of claim 1, 2, 3, or 4 wherein the purpose of the study is to compare the effects, or relative effects, of different contact lens materials and/or wearing periods.

13. The method of claim 1, 2, 3, or 4 wherein study results are to be submitted to the United States Food and Drug Administration for approval to market a product or service or to support approval of product or service marketing claims.

14. The method of claim 1, 2, 3, or 4 and wherein the purpose of the study is to identify one or more mammals with no substantive change or changes in ocular defenses or health associated with a product or service under study.

15. The method of claim 1, 2, 3, or 4 wherein the purpose of the study is to identify one or more mammals with an improvement in ocular defenses or health associated with a product or service under study as represented by a substantive:
(a) increase in tear constituent with antioxidant effect; or
(b) increase in tear constituent with antimicrobial effect; or
(c) decrease in inflammatory mediator or byproduct tear constituent; or
(d) decrease in product or byproduct of cellular damage tear constituent.

16. The method of claim 1, 2, 3, or 4 wherein the purpose of the study is to evaluate the relative risk of one or more mammals suffering adverse events or complications associated with a product or service under study.

17. The method of claim 1, 2, 3, or 4 wherein a tear fluid sample is taken directly from the eye of a mammal using a micropipette.

18. The method of claim 1, 2, 3, or 4 wherein a tear fluid sample is taken from the eye of a mammal via absorption into or onto a contact lens, bandage lens, collagen lens, absorbent paper or other material placed in contact with a mammal's tears.

19. The method of claim 1, 2, 3, or 4 wherein a tear constituent is measured in or after extraction from contact lens, bandage lens, collagen lens, absorbent paper or other material.

20. The method of claim 1, 3, or 4 wherein measurement of a tear constituent is taken after an ophthalmic surgical procedure; wear of an ophthalmic device; use of a product with ophthalmic effect; or exposure to a new or proposed application of a device or product with ophthalmic effect.

21. The method of claim 1, 2, 3, or 4 wherein a tear constituent has antioxidant effect.

22. The method of claim 21 wherein the tear constituent is ascorbic acid, uric acid, lactoferrin, alpha-tocopherol, superoxide dismutase, catalase, or glutathione peroxidase.

23. The method of claim 1, 2, 3, or 4 wherein a tear constituent has antimicrobial effect.

24. The method of claim 23 wherein the tear constituent is lactoferrin, lysozyme, or secretory phospholipase A2.

25. The method of claim 1, 2, 3, or 4 wherein a tear constituent is an inflammatory mediator or byproduct.

26. The method of claim 25 wherein the tear constituent is an immunoglobulin, leukotriene, cytokine, or albumin.

27. The method of claim 26 wherein the immunoglobulin tear constituent is IgA, total sIgA, microbial-specific sIgA, IgE, or IgM.

28. The method of claim 26 wherein the leukotriene tear constituent is LTB₄ or LTC₄.

29. The method of claim 1, 2, 3, or 4 wherein a tear constituent is a product or byproduct of cellular damage.

30. The method of claim 29 wherein the product or byproduct of cellular damage tear constituent is lactate dehydrogenase, malate dehydrogenase, fibronectin, or plasmin.

31. The method of claim 1, 2, 3, or 4 wherein measurement of a tear constituent is performed by an enzyme-linked immunosorbent assay.

32. The method of claim 31 wherein the enzyme-linked immunosorbent assay is specifically designed for measurement of one or more tear constituents.

33. The method of claim 31 wherein the enzyme-linked immunosorbent assay is part of a test kit designed for measurement of one or more tear constituents.

34. The method of claim 1, 2, 3, or 4 wherein measurement a tear constituent is performed by dipstick.

35. The method of claim 34 wherein the dipstick is specifically designed for measurement of one or more tear constituents.

36. The method of claim 34 wherein the dipstick is part of a test kit designed for measurement of one or more tear constituents.

37. The method of claim 1, 2, 3, or 4 wherein the measured level, or change in level, of a tear constituent is mathematically adjusted, statistically described, graphically represented, or otherwise translated, converted or modified.

38. The method of claim 1, 2, 3, or 4 wherein the measured level, or change in level, of a tear constituent before or after mathematical adjustment, statistical description, graphical representation, or other translation, conversion or modification is combined with the measured level, or change in level, of a different tear constituent before or after mathematical adjustment, statistical description, graphical representation, or other translation, conversion or modification.

39. The method of claim 1, 2, 3, or 4 wherein the measured level, or change in level, of a tear constituent before or after mathematical adjustment, statistical description, graphical representation, or other translation, conversion or modification is combined with a characterization, evaluation, or finding from application of conventional ophthalmic screening or diagnostic methods before or after mathematical adjustment, statistical description, graphical representation, or other translation, conversion or modification.

40. The method of claim 1 or 3 wherein the measured level, or change in level, of a tear constituent is compared to a previous or subsequent measured level, or change in level, of the same or equivalent tear constituent from the same mammal.

41. The method of claim 1, 2, or 3 wherein the measured level, or change in level, of a tear constituent from a mammal is compared to a measured level, or change in level, of the same or equivalent tear constituent obtained from one or more different mammals.

42. The method of claim 40 or 41 wherein comparison is made using similar mathematical, statistical, graphical, or other descriptions or representations of the measured level, or change in level, of a tear constituent.

43. The method of claim 40 or 41 wherein comparison indicates a deterioration in ocular defenses or health, as represented by a substantive:
(a) decrease in a tear constituent with antioxidant effect; or
(b) decrease in a tear constituent with antimicrobial effect; or
(c) increase in a inflammatory mediator or byproduct tear constituent; or
(d) increase in a product or byproduct of cellular damage tear constituent.

44. The method of claim 40 or 41 wherein comparison indicates an improvement in ocular defenses or health, as represented by a substantive:
(a) increase in a tear constituent with antioxidant effect; or
(b) increase in a tear constituent with antimicrobial effect; or
(c) decrease in a inflammatory mediator or byproduct tear constituent; or
(d) decrease in a product or byproduct of cellular damage tear constituent.

45. The method of claim 14, 15, 43 or 44 wherein substantive is defined as a 20% or greater increase or decrease in measured level or change in level.

46. The method of claim 14, 15, 43 or 44 wherein the definition of substantive is determined by the purposes of the testing.

47. The method of claim 41 wherein comparison is made to one or more mammals selected to be representative of mammals: generally in a population; in good health in a specific population; from specific demographic groups; suffering from specific diseases or disorders; with common known response; or otherwise, according to the purposes of comparison.

48. The method of claim 47 wherein a group of mammals used for comparative purposes is large enough to represent a similar population-wide group with greater than 90% confidence.
